(19) United States
(12) Patent Application Publication
(10) Pub. No.: US 2024/0181144 A1
(43) Pub. Date: Jun. 6, 2024

(54) DIALYSIS SYSTEM HAVING REDUCED VALVE NOISE AND VALVE POSITION DETECTION

(71) Applicants: BAXTER INTERNATIONAL INC., Deerfield, IL (US); BAXTER HEALTHCARE SA, Glattpark (Opfikon) (CH)

(72) Inventor: John Zafiris, Hawthorn Woods, IL (US)

(21) Appl. No.: 18/528,144

(22) Filed: Dec. 4, 2023

Related U.S. Application Data
(60) Provisional application No. 63/429,782, filed on Dec. 2, 2022.

(51) Int. Cl. A61M 1/16; A61M 39/24

(52) U.S. Cl. CPC A61M 1/1621; A61M 39/24; A61M 2205/50

(57) ABSTRACT

Systems and methods are disclosed for reducing valve noise and detecting a valve position in medical fluid systems. One method includes transmitting, by a microcontroller, a control signal for closing a valve via pulse width modulation (PWM) signals. The valve is configured to control a fluid flow in the medical fluid system. The valve may be open at least before the control signal is transmitted. The valve comprises a housing, a solenoid coil, and a plunger. The method further includes applying power to the valve to measure voltage across the valve and monitoring, based on a sense resistor, the voltage across the valve over a measurement interval. The measurement interval terminates when the voltage reaches a predetermined threshold voltage. The method also includes comparing the measurement interval to a reference interval for a normally functioning closed valve and generating, based on the comparison, an assessment of the valve position.

### PRIORITY CLAIM

This application claims priority to and the benefit as a non-provisional application of U.S. Provisional Patent Application No. 63/429,782, filed Dec. 2, 2022, the entire contents of which are hereby incorporated by reference and relied upon.

### TECHNICAL FIELD

The present disclosure relates generally to medical fluid treatments, and in particular to dialysis fluid treatments that use valves for medical fluid control.

### BACKGROUND

Due to various causes, a person's renal system can fail. Renal failure produces several physiological derangements. For instance, it is no longer possible to balance water and minerals or to excrete daily metabolic load. Toxic end products of metabolism, such as, urea, creatinine, uric acid, and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for the replacement of kidney functions is critical to many people because the treatment is lifesaving.

One type of kidney failure therapy is hemodialysis (“HD”), which in general uses diffusion to remove waste products from a patient's blood. A diffusive gradient occurs across a semi-permeable dialyzer between blood and an electrolyte solution called dialysate or dialysis fluid to cause diffusion.

Hemofiltration (“HF”) is an alternative renal replacement therapy that relies on a convective transport of toxins from a patient's blood. HF is accomplished by adding substitution or replacement fluid to an extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, thereby providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. Bo comparison. HHD can take place overnight or during the day while the patient relaxes, works, or is otherwise productive.

Another type of kidney failure therapy is peritoneal dialysis (“PD”), which infuses a dialysis solution, also called dialysis fluid, into a patient's peritoneal chamber via a catheter. The dialysis fluid contacts a peritoneal membrane in a patient's peritoneal chamber. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, thereby removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, where the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill, and dwell cycles. APD machines, however, perform the cycles automatically, typically while a patient sleeps. APD machines free patients from having to manually perform the treatment cycles and from having to transport supplies during the day. APD machines connect fluidly to an implanted catheter, a source or bag of fresh dialysis fluid, and a fluid drain. APD machines pump fresh dialysis fluid from a dialysis fluid source, through the catheter and into the patient's peritoneal chamber. APD machines also allow for the dialysis fluid to dwell within the chamber and for the transfer of waste, toxins, and excess water to take place. The source may include multiple liters of dialysis fluid including several solution bags.

APD machines pump used or spent dialysate from the patient's peritoneal cavity, though the catheter, to drain. As with the manual process, several drain, fill, and dwell cycles occur during dialysis. A “last fill” may occur at the end of the APD treatment. The last fill fluid may remain in the peritoneal chamber of the patient until the start of the next treatment, or may be manually emptied at some point during the day.

Each of the above-identified dialysis modalities, except for CAPD (which typically does not involve machinery), uses automated valves to control whether dialysis fluid, blood, or other fluid is able to flow or not flow. The valves also control the direction of fluid flow, such as where the fluid comes from or the destination to which the fluid flows. Different types of valves are used in dialysis system. One type of valve is typically used with a disposable cassette having a hard plastic part defining fluid flow paths and valve seats and one or more flexible membrane covering one or more side of the hard plastic part. The disposable cassette is typically loaded into a dialysis machine or cycler, which is able to close designated parts of the one or more plastic sheet against the valve seats to block fluid flow and to force or allow the plastic to move away from the valve seats to allow fluid flow.

Another type of automated valve is a solenoid pinch valve that instead pinches closed a tube carrying dialysis fluid, blood, or other fluid to block fluid flow. Here, a hard plastic disposable cassette is not needed, saving cost. There are generally two types of pinch valves, solenoid pinch valves and motorized pinch valves. A further type of automated valve is a solenoid plunger valve. The solenoid plunger valve uses a plunger (e.g., a metal slug that moves through a solenoid coil via electromagnetic induction) to move a lever that is pressed against a seat to stop fluid flow (or move the lever to press the seat to cause fluid flow). One problem with solenoid pinch valves and solenoid plunger valves (collectively referred to herein as “solenoid valves”) is noise. For example, solenoid plunger valves generally energize a coil that moves a plunger within a housing. The plunger may be moved while the coil is energized to allow the tube to open for fluid flow. When energy is removed from the coil, a compressed spring is allowed to push the plunger in an opposite direction to occlude the tube against a stop or wall located on the opposing end of the tube. The plunger moving in either direction encounters an end-of-travel that involves the plunger contacting a fixed surface either directly or with the tube in between. Noise is created when the plunger contacts the end-of-travel. Noise from solenoid valves may disturb a patient and be problematic. This is especially true for an APD treatment, which typically occurs at night while a patient sleeps.

Another issue with solenoid valves is knowing that the valve has opened when energized (or closed when de-energized). That is, knowing that the energizing of the coil has actually moved the plunger (e.g., in a solenoid plunger valve) or actually removes the pinching of a tube (e.g., in a solenoid pinch valve) so that it no longer occludes a tube or fluid flow. Assuming that a valve is open when it may not actually be open creates an undesirable situation.

For each of the above issues, an improved way to operate solenoid valves is needed.

### SUMMARY

The present disclosure sets forth methodologies for operating solenoid valves for use in medical fluid systems, such as an automated peritoneal dialysis (“PD”) system, which improves the usability of the valves. While the present system is described primarily in connection with PD, the improved solenoid valve operation of the present disclosure applies to machines used for any dialysis modality described herein, such as online HD, HF, HDF, acute HD, HF, and HDF. The improved solenoid valve operation of the present disclosure also applies to any medical fluid system in which a treatment fluid flow or a patient fluid flow is controlled via one or more valve.

In a PD example, the system includes a PD machine or cycler. The PD machine is described herein primarily as a durable system that attempts to limit disposable waste as much as possible, e.g., via the use of electromechanical piston pumps that pump medical or PD fluid through a body of a pump. The PD fluid pump may also be an electromechanically driven gear, peristaltic, or centrifugal pump. In a further alternative embodiment, a pneumatically driven PD fluid pump may be employed. Any of the above pumping scenarios may be used in combination with the electromechanically actuated solenoid valves of the present disclosure. The PD machine or cycler is in one embodiment capable of delivering fresh, heated PD fluid to a patient at, for example, 14 kPa (2.0 psig) or higher. The PD machine is capable of removing used PD fluid or effluent from the patient at, for example, −9 kPa (−1.3 psig) or an even greater negative pressure. Fresh PD fluid delivered to the patient may be first heated to a body fluid temperature, e.g., 37° C.

The PD machine or cycler also includes a plurality of valves, any one, or more, or all of which may be solenoid valves. The solenoid valves discussed herein may be of any variety or type. For example, one type of solenoid valve uses an internal fluid pathway that is either open or closed depending on whether the coil is energized. This type of solenoid valve is well suited for durable or reusable versions of the PD machine or cycler. Another type of solenoid valve operates by unclosing or closing a flexible tube depending on whether the coil is energized. This type of solenoid valve is well suited for versions of the PD machine or cycler operating with a disposable set but may also be used with a durable version of the PD machine or cycler, which has internal flexible tubing for operating with the solenoid valves.

The system and associated methodology of the present disclosure automatically determine the position or state of a solenoid valve, which may be used to detect a stuck valve and other valve faults. Automatically detecting valve position allows for automatic self-calibration of the solenoid valve hardware in addition to detecting faults. The self-calibration also aids in implementing the noise reduction methodology discussed herein. The system includes electrical hardware and software and is configured to control an electromechanical device, e.g., a solenoid valve.

### Noise Reduction

The PD machine or cycler of the present system operates with solenoid valves under control of a control unit. The control unit in various embodiments controls the movement of the solenoid valve to minimize an amount of sound that occurs at impact during activation and deactivation of the valve. The control includes the use of a PWM drive waveform delivered via a microcontroller programmed to cause a PWM duty cycle to increase from zero to one-hundred percent (close valve) and to decrease from one-hundred to zero percent over a curved profile as opposed to an instantaneous jump or drop-off. The curved profiles become more horizontal toward the end of plunger travel, thereby lessening the impact force created by the valve lever and reducing the sound or noise generated in connection with the end of opening or closing the solenoid valves. The curved profiles are implemented electrically at the valve coils via a metal oxide semiconductor field effect transistor (“MOSFET”) and a diode, in one embodiment.

### Determining the Position of the Solenoid Valve

As discussed herein, solenoid valves generally open by energizing a coil that moves a plunger within a housing of the solenoid valve (e.g., as in a solenoid plunger valve) and/or releases a pinched status of a tube (e.g., as in a solenoid pinch valve). There is a desire and need to assess the position of the solenoid valve of a medical fluid machine or cycler (e.g., a PD, an HD, an HF, an HDF, and/or a CRRT machine or cycler) to ensure that the valve is not stuck or otherwise compromised. In addition, for the noise reduction system and associated methodology disclosed herein, the valve lever position detection is useful so that it is ensured that the noise reducing PWM drive waveforms are commenced when the valve lever is in the fully open or fully closed position as needed.

In an embodiment, an analog signal is delivered along a position detection line extending from a point electrically upstream from a MOSFET to a multiplexer that allows multiple solenoid valves to be analyzed sequentially. An output from the multiplexer is delivered to a comparator that compares each valve's analog signal to a threshold value. The comparator outputs a signal when the valve's analog signal reaches the threshold value. The comparator outputs the signal to a microcontroller, which is programmed to determine a valve lever position for a particular valve from timing of when the signal is received.

In light of the disclosure set forth herein, and without limiting the disclosure in any way, in a first aspect of the present disclosure, which may be combined with any other aspect, or portion thereof, a medical fluid system is disclosed, which includes a driving circuit, a valve, and a microcontroller. The driving circuit is configured to control the valve via pulse width modulation (PWM) signals, in response to control signals received from the microcontroller. The valve is configured to control a fluid flow in the medical fluid system. The valve includes a housing, a solenoid coil, and a plunger. The valve is configured to activate a flow of fluid through a tube by applying, via the driving circuit, voltage to the solenoid coil to move the plunger within the housing.

In a second aspect of the present disclosure, which may be combined with any other aspect, or portion thereof, the instructions, when executed by the processor, further cause the processor to apply, via the driving circuit, power to the valve to measure a voltage across the valve, monitor, via the driving circuit based on a sense resistor associated with the valve, the voltage across the valve over a measurement interval, where the measurement interval terminates when the voltage reaches a predetermined threshold voltage, compare the measurement interval to a reference interval for a normally functioning closed valve, and generate, based on the comparison of the measurement interval to the reference interval, an assessment of the valve position.

In a third aspect of the present disclosure, which may be combined with any other aspect, or portion thereof, the valve is further configured to: close the flow of medical fluid through the tube by un-applying, via the driving circuit, the voltage to the solenoid coil to move the plunger in an opposite direction from the housing to occlude the tube. Furthermore, the instructions, when executed by the processor, further cause the processor to: transmit, to the driving circuit, a second control signal causing the driving circuit to ramp down the PWM signal to the solenoid coil to a duty cycle of 0%. The ramping down of the PWM signal causes the plunger to move in the opposite direction and in a manner in which sound generated by the corresponding plunger is reduced in comparison to operation without the PWM signal.

In a fourth aspect of the present disclosure, which may be combined with any other aspect, or portion thereof, the instructions, when executed by the processor, further cause the processor to: apply, via the driving circuit, second power to the valve to measure second voltage across the valve; monitor, based on a sense resistor associated with the valve, and via the driving circuit, second voltage across the valve over a second measurement interval, wherein the second measurement interval terminates when the voltage reaches the predetermined threshold voltage; compare the second measurement interval to a second reference interval for a normally functioning open valve; and generate, based on the comparison of the second measurement interval to the second reference interval, a second assessment of the valve position.

In a fifth aspect of the present disclosure, which may be combined with any other aspect, or portion thereof, a method of determining valve position in a medical fluid system is disclosed. The method includes transmitting, by a microcontroller having a processor, a control signal for closing a valve, wherein the valve is configured to control a fluid flow in the medical fluid system, wherein the valve is open at least before the control signal is transmitted, and wherein the valve comprises a housing, a solenoid coil, and a plunger; applying, via a driving circuit, power to the valve to measure a voltage across the valve; monitoring, via the driving circuit based on a sense resistor associated with the valve, the voltage across the valve over a measurement interval, wherein the measurement interval terminates when the voltage reaches a predetermined threshold voltage; comparing the measurement interval to a reference interval for a normally functioning closed valve; and generating, based on the comparison, an assessment of the valve position.

In a sixth aspect of the present disclosure, which may be combined with any other aspect, or portion thereof, the method further comprises, prior to applying the power to the valve setting power level to zero for a predetermined period of time to cause desaturation of the solenoid coil over the predetermined period of time.

In a seventh aspect of the present disclosure, which may be combined with any other aspect, or portion thereof, the predetermined period of time is 5 milliseconds.

In an eight aspect of the present disclosure, which may be combined with any other aspect, or portion thereof, monitoring the voltage across the valve comprises receiving, from a comparator connected to the driving circuit, an indication of when the voltage reaches the predetermined threshold voltage.

In a ninth aspect of the present disclosure, which may be combined with any other aspect, or portion thereof, the method further comprises transmitting, by the microcontroller, a second control signal for opening the valve; applying, via the driving circuit, second power to the valve to measure second voltage across the valve; monitoring, based on the sense resistor, and via the driving circuit, second voltage across the valve over a second measurement interval, wherein the second measurement interval terminates when the second voltage reaches a second predetermined threshold voltage; compare the second measurement interval to a second reference interval for a normally functioning open valve; and generate, based on the comparison between the second measurement interval with the second reference interval, a second assessment of the valve position.

In a tenth aspect of the present disclosure, which may be combined with any other aspect, or portion thereof, any of the features, functionality and alternatives described in connection with any one or more of FIGS. 1 to 16 may be combined with any of the features, functionality and alternatives described in connection with any other of FIGS. 1 to 16.

In light of the above aspects and present disclosure set forth herein, it is an advantage of the present disclosure to provide a medical fluid system having improved solenoid valve operation.

It is another advantage of the present disclosure to provide a medical fluid system having solenoid valve methodology that reduces noise created by valve operation.

It is a further advantage of the present disclosure to provide a medical fluid system having solenoid valve methodology that enables a position or state of a solenoid valve to be determined, which may be used to detect when the solenoid valve is stuck or has other faults.

It is yet another advantage of the present disclosure to provide a medical fluid system having solenoid valve methodology that enables a position or state of a solenoid valve to be determined using information from the solenoid valve itself, thereby reducing hardware needed.

It is yet a further advantage of the present disclosure to provide a medical fluid system having solenoid valve methodology that enables a solenoid valve to be self-calibrated.

It is still another advantage of the present disclosure to provide a medical fluid system having solenoid valve methodology that enables a position or state of a solenoid valve to be determined with high precision and high resolution (e.g., better than 0.01 millimeter).

It is still another advantage of the present disclosure to provide a medical fluid system having solenoid valve methodology that uses common, low cost electrical parts.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view of one embodiment for an automated PD system having a solenoid valve, in accordance with a non-limiting embodiment of the present disclosure.

FIG. 2 is a sectioned elevation view of one embodiment for a two-way valve useable with the systems and associated methodology of the present disclosure, in accordance with a non-limiting embodiment of the present disclosure.

FIG. 3 is a sectioned elevation view of one embodiment for a three-way valve useable with the systems and associated methodology of the present disclosure, in accordance with a non-limiting embodiment of the present disclosure.

FIG. 4 is an example solenoid valve driving circuit for the systems and associated methodology of the present disclosure, which may be used to lessen operational noise associated with solenoid valves, in accordance with a non-limiting embodiment of the present disclosure.

FIG. 5 is a pulse width modulation (“PWM”) plot illustrating a noisy valve that does not use the PWM drive waveform of the present disclosure, in accordance with a non-limiting embodiment of the present disclosure.

FIG. 6 is a PWM plot illustrating a quiet valve using the PWM drive waveform of the present disclosure for both energization and de-energization, in accordance with a non-limiting embodiment of the present disclosure.

FIG. 7 is an expanded example solenoid valve driving circuit for multiple valves, which may be used to lessen operational noise associated with solenoid valves and which multiplexes outputs from multiple valve circuits, in accordance with a non-limiting embodiment of the present disclosure.

FIG. 8 is an electrical schematic illustrating the driving circuit for determining a position of a valve lever in a given solenoid valve, according to an example embodiment of the present disclosure.

FIG. 9 is a graphical output from a valve lever position test configuration, which illustrates the position of the valve based on inductance, in accordance with non-limiting embodiments of the present disclosure.

FIG. 10 is a data output from a valve lever position test configuration, which illustrates the position of a plunger in the valve over time, in accordance with a non-limiting embodiment of the present disclosure.

FIG. 11 illustrates various waveforms from a valve lever position test, which illustrate measured voltage over time to determine the position of the valve when a valve is open and when a valve is closed, in accordance with non-limiting embodiments of the present disclosure.

FIG. 12 illustrates an averaged waveform from multiple valve lever position tests, which illustrate a characteristic transition point for that valve, in accordance with a non-limiting embodiment of the present disclosure.

FIGS. 13 and 14 illustrate valve lever 170, 210 position test data taken after a gate drive has been desaturated, in accordance with non-limiting embodiments of the present disclosure.

FIG. 15 illustrate valve lever position test data recorded showing the valve lever being stuck closed, the valve lever acting normally, and the valve lever stuck open, when the valve is commanded to transition from an open to a closed state, in accordance with non-limiting embodiments of the present disclosure.

FIG. 16 illustrate valve lever position test data recorded showing the valve lever being stuck closed, the valve lever acting normally, and the valve lever stuck open, when the valve is commanded to transition from a closed to an open state, in accordance with non-limiting embodiments of the present disclosure.

### DETAILED DESCRIPTION

### System Overview

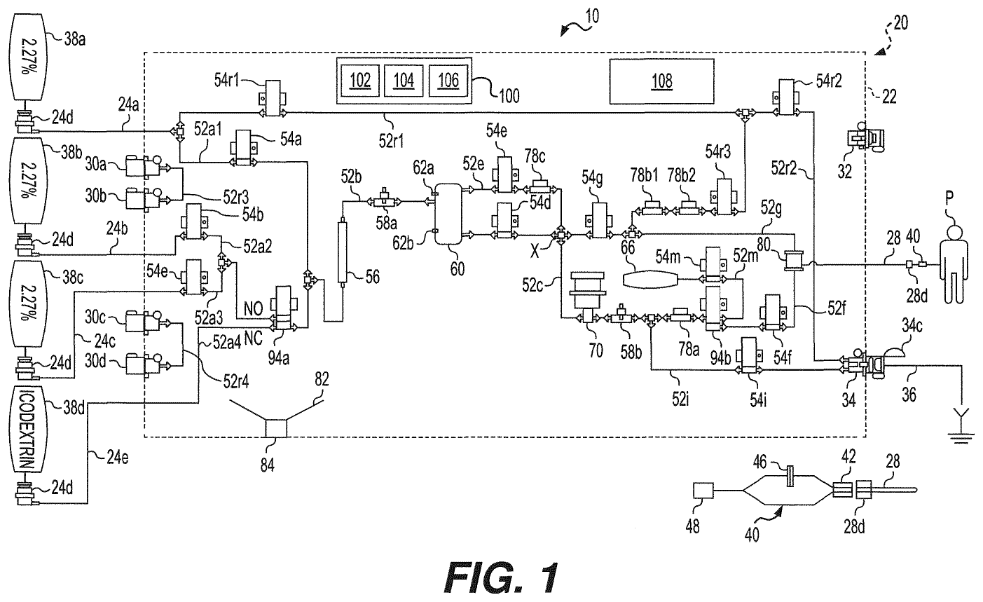

Referring now to the drawings and in particular to FIG. 1, an example system 10 including the solenoid valve operation of the present disclosure is illustrated. System 10 includes a dialysis machine 20, such as an automated peritoneal dialysis (“PD”) machine, and a control unit 100 having one or more processor 102, one or more memory 104, video controller 106, and user interface 108. Control unit 100 controls all electrical fluid flow and heating components of system 10 and receives outputs from all sensors of system 10. System 10 in the illustrated embodiment includes durable and reusable components that contact medical fluid, such as PD fluid, which necessitates that PD machine or cycler 20 be disinfected between treatments, e.g., via heat disinfection.

System 10 in FIG. 1 includes an inline resistive heater 56, reusable supply lines or tubes 52al to 52a4 and 52b, air trap 60 operating with respective upper and lower level sensors 62a and 62b, air trap valve 54d, vent valve 54e located along vent line 52e, reusable line or tubing 52c, dialysis fluid pump 70, temperature sensors 58a and 58b, pressure sensors 78a, 78b1, 78b2, and 78c, reusable patient tubing or lines 52f and 52g having respective valves 54f and 54g, dual lumen reusable patient line 28, hose reel 80 for retracting patient line 28, reusable drain tubing or line 52i extending to drain line connector 34 and having a drain line valve 54i, and first and second reusable recirculation disinfection tubing or lines 52r1 and 52r2 operating with respective disinfection valves 54r1 and 54r2. A third recirculation or disinfection tubing or line 52r3 extends between disinfection connectors 30a and 30b for use during disinfection. A fourth recirculation or disinfection tubing or line 52r4 extends between disinfection connectors 30c and 30d for use during disinfection.

System 10 further includes PD fluid containers or bags 38a to 38c (e.g., holding the same or different formulations of PD fluid), which connect to distal ends 24d of reusable PD fluid lines 24a to 24c, respectively. System 10d further includes a fourth PD fluid container or bag 38d that connects to a distal end 24d of reusable PD fluid line 24e. Fourth PD fluid container or bag 38d may hold the same or different type (e.g., icodextrin) of PD fluid than provided in PD fluid containers or bags 38a to 38c. Reusable PD fluid lines 24a to 24c and 24e extend in one embodiment through apertures (not illustrated) defined or provided by housing 22 of cycler 20.

System 10 in the illustrated embodiment includes four disinfection connectors 30a to 30d for connecting to distal ends 24d of reusable PD fluid lines 24a to 24c and 24e, respectively, during disinfection. System 10 also provides patient line connector 32 that includes an internal lumen, e.g., a U-shaped lumen, which directs fresh or used dialysis fluid from one PD fluid lumen of dual lumen reusable patient line 28 into the other PD fluid lumen. Reusable supply tubing or lines 52al to 52a4 communicate with reusable supply lines 24a to 24c and 24e, respectively. Reusable supply tubing or lines 52al to 52a3 operate with valves 54a to 54c, respectively, to allow PD fluid from a desired PD fluid container or bag 38a to 38c to be pulled into cycler 20. Three-way valve 94a in the illustrated example allows for control unit 100 to select between (i) 2.27% (or other) glucose dialysis fluid from container or bag 38b or 38c and (ii) icodextrin from container or bag 38d. In the illustrated embodiment, icodextrin from container or bag 38d is connected to the normally closed port of three-way valve 94a.

FIG. 1 also illustrates that system 10 includes and uses disposable filter set 40, which communicates fluidly with the fresh and used PD fluid lumens of dual lumen reusable patient line 28. Disposable filter set 40 includes a disposable connector 42 that connects to distal end 28d of reusable patient line 28. Disposable filter set 40 includes a connector 48 that connects to the patient's transfer set. Disposable filter set 40 further includes a sterilizing grade filter membrane 46 that further filters fresh PD fluid.

System 10 is constructed, in one embodiment, such that drain line 52i during filling is fluidly connected downstream from dialysis fluid pump 70. In this manner, if drain valve 54i fails or somehow leaks during a patient fill of patient P, fresh PD fluid is pushed down disposable drain line 36 instead of used PD fluid potentially being pulled into pump 70. Disposable drain line 36 is, in one embodiment, removed for disinfection, while drain line connector 34 is capped via a cap 34c.

System 10 further includes a leak detection pan 82 located at the bottom of housing 22 of cycler 20 and a corresponding leak detection sensor 84 outputting to control unit 100. In the illustrated example, system 10 is provided with an additional pressure sensor 78c located upstream of dialysis fluid pump 70, which allows for the measurement of the suction pressure of pump 70 to help control unit 100 more accurately determine pump volume. Additional pressure sensor 78c in the illustrated embodiment is located along vent line 52e, which may be filled with air or a mixture of air and PD fluid, but which should nevertheless be at the same negative pressure as PD fluid located within PD fluid line 52c.

System 10 in the example of FIG. 1 includes redundant pressure sensors 78b1 and 78b2, the output of one of which is used for pump control, as discussed herein, while the output of the other pressure sensor is a safety or watchdog output to make sure the control pressure sensor is reading accurately. Pressure sensors 78b1 and 78b2 are located along a line including a third recirculation valve 54r3. In still a further example, system 10 may employ one or more cross, marked via an X in FIG. 1, which may (i) reduce the overall amount and volume of the internal, reusable tubing. (ii) reduce the number of valves needed, and (iii) allow the portion of the fluid circuitry shared by both fresh and used PD fluid to be minimized.

System 10 in the example of FIG. 1 further includes a source of acid, such as a citric acid container or bag 66. Citric acid container or bag 66 is in selective fluid communication with second three-way valve 94b via a citric acid valve 54m located along a citric acid line 52m. Citric acid line 52m is connected in one embodiment to the normally closed port of second three-way valve 94b, so as to provide redundant valves between citric acid container or bag 66 and the PD fluid circuit during treatment. The redundant valves ensure that no citric (or other) acid reaches the treatment fluid lines during treatment. Citric (or other) acid is instead used during disinfection.

It should be appreciated that system 10 is not required to (i) be a dialysis system, or (ii) use redundant or durable components that are disinfected between uses to employ the sensor thermoelectric heating of the present disclosure. System 10 may instead be any type of medical fluid system and may employ a disposable set having a disposable pumping portion that contacts the corresponding medical fluid. In the primary example described herein, the solenoid valves are described as operating with PD machine or cycler 20.

Any one or more or all of valves 54a to 54h, 54m, and 54r1 to 54r4, 94a and 94b may be a solenoid valve, which may be of a type of that uses an internal fluid pathway that is either open or closed depending on whether the coil is energized. This type of solenoid valve is well suited for durable or reusable versions of the PD machine or cycler. Another type of solenoid valve for valves 54a to 54h, 54m, and 54r1 to 54r4 operates by unclosing or closing a flexible tube depending on whether the coil is energized. This type of solenoid valve is well suited for versions of the PD machine or cycler operating with a disposable set but may also be used with a durable version of the PD machine or cycler, which would have internal flexible tubing for operating with the solenoid valves.

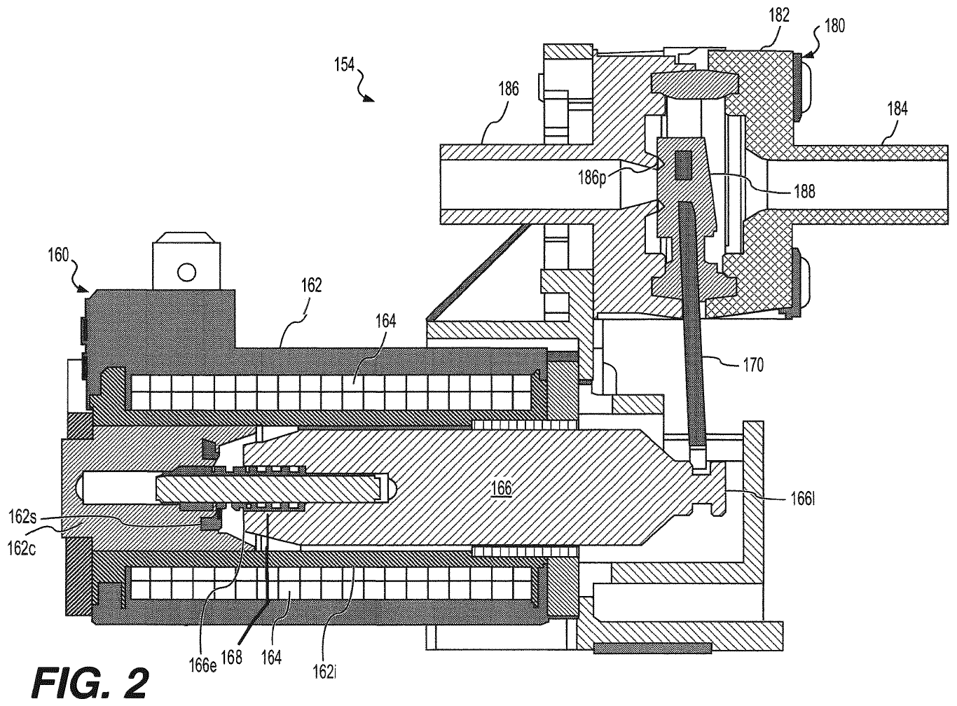

Referring now to FIG. 2, one suitable two-way solenoid valve 154 for two-way valves 54a to 54h, 54m, and 54r1 to 54r4 is illustrated. Valve 154 of FIG. 2 is of a type that uses an internal fluid pathway that is either open or closed depending on whether the coil is energized. Valve 154 includes two primary sections, namely, a solenoid section 160 and a valve section 180. Solenoid section 160 includes a solenoid housing 162. Solenoid housing 162 supports a coil 164, which extends around an inner wall 162i of housing 162, and which is energized to move or translate a solenoid plunger 166. A compression spring 168 is provided and is positioned so as to bias plunger 166 into a closed position when energy is removed from coil 164.

FIG. 2 illustrates that plunger 166 has a contacting end 166e. Also, a core portion 162c of solenoid housing 162 is provided with a stop 162s. When coil 164 is energized, a magnetic field is induced, causing solenoid plunger 166 to translate within the inner wall 162i of housing 162 from right to left, such that contacting end 166e of plunger 166 abuts stop 162s to provide an end-of-travel for plunger 166 in a valve open position. The abutting contact of end 166e with stop 162s causes noise, which may become problematic for the PD patient, especially when the patient is trying to sleep. Described herein is structure and associated methodology to help reduce the noises cause by the abutting contact of end 166e with stop 162s.

FIG. 2 illustrates two-way solenoid valve 154 in a closed or no fluid flow condition. Here, coil 164 is not energized, such that compression spring 168 pushes contacting end 166e of plunger 166 away from the stop 162s provided at core portion 162c of solenoid housing 162. A lever end 1661 of plunger 166 is translated such that a lever 170, held rotatably at the lever end 1661 of plunger 166, is tilted so as to close a fluid pathway located within valve section 180 of valve 154.

Valve section 180 of valve 154 includes a valve housing 182. Valve housing 182 defines a fluid inlet 184 and a fluid outlet 186. The portion of lever 170, extending into valve housing 182, is fitted with a membrane or stopper 188, which may be made of a medically safe compressible (sealable) rubber, such as silicone. In the closed position of FIG. 2, where coil 164 is not energized and compression spring 168 is extended, lever 170 pivots membrane or stopper 188 so as to contact and seal closed an inner, e.g., beveled port 186p of outlet 186, thereby preventing fluid flow. When coil 164 is energized, plunger 166 moves to the left so as to compress spring 168 and pivot lever 170, such that membrane or stopper 188 moves away from beveled port 186p of outlet 186 and stops at a substantially vertical position. Here, fluid, such as water or PD fluid is able to flow from fluid inlet 184, around membrane or stopper 188, and through fluid outlet 186.

When valve 154 is closed and membrane or stopper 188 is sealed against beveled port 186p of fluid outlet 186, the fluid pressure downstream from outlet 186 is less than the fluid pressure upstream of fluid inlet 184. The pressure delta helps to seal membrane or stopper 188 against beveled port 186p, such that compression spring 168 does not need to supply a force needed (or all of the force needed) to keep the membrane or stopper sealed against the beveled port. The main function of compression spring 168 is to translate plunger 166 when coil 164 is de-energized. It should be appreciated, however, that the pressure delta that helps to seal membrane or stopper 188 against beveled port 186p when valve 154 is to be closed, also fights against the magnetic force induced when coil 164 is energized. Described herein is structure and associated functionality for ensuring that valve 154 is properly opened when it is commanded to be open.

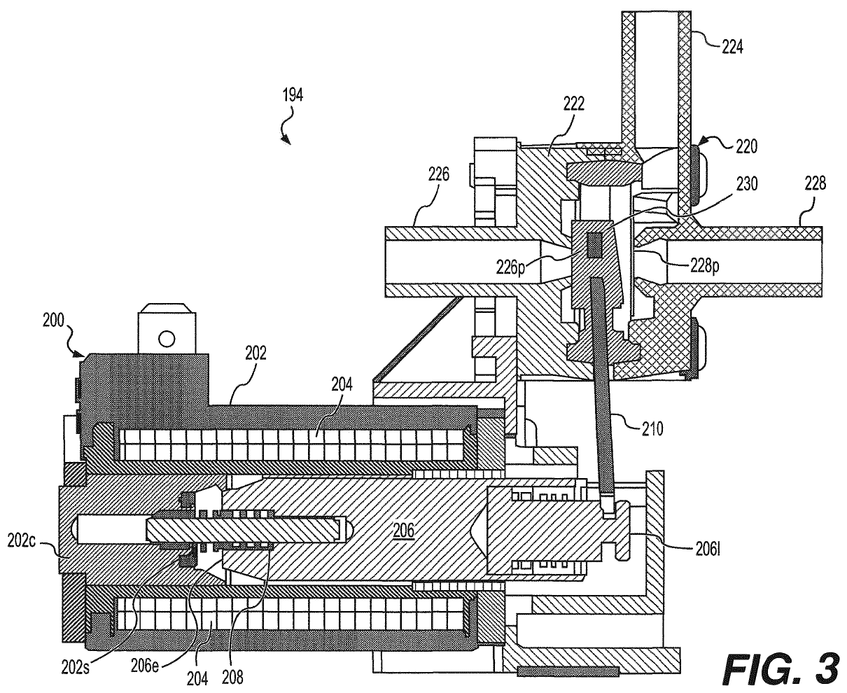

Referring now to FIG. 3, one suitable three-way solenoid valve 194 for three-way valves 94a, 94b is illustrated. Valve 194 of FIG. 3 is of a type of that uses an internal fluid pathway that is either open or closed depending on whether the coil is energized. Valve 194, like valve 154, includes two primary sections, namely, a solenoid section 200 and a valve section 220. Solenoid section 200 is basically the same as solenoid section 160 of valve 154. Solenoid section includes a solenoid housing 202 that supports a coil 204, which is energized to move or translate a solenoid plunger 206. A compression spring 208 is provided and is positioned so as to bias plunger 206 into a closed position when energy is removed from coil 164. Plunger 206 has a contacting end 206e, which abuts a stop 202s that is provided at a core portion 202c of solenoid housing 202 when coil 204 is energized. The abutting contact of end 206e with stop 202s causes noise, which may become problematic for a PD patient, especially when the patient is trying to sleep. Described herein is structure and associated methodology to help reduce the noises cause by the abutting contact of end 206e with stop 202s.

FIG. 3 illustrates three-way solenoid valve 194 in a normally closed condition. Here, coil 204 is not energized, such that compression spring 208 pushes contacting end 206e of plunger 206 away from the stop 202s provided at core portion 202c of solenoid housing 202. A lever end 2061 of plunger 206 is translated such that a lever 210, held rotatably at the lever end 2061, of plunger 206 is tilted so as to close a fluid pathway located within valve section 220 of valve 194.

Valve section 220 is where three-way valve 194 differs from two-way valve 154. Valve section 220 includes a valve housing 222 defining a fluid inlet 224, a normally closed fluid outlet 226, and a normally open fluid outlet 228. The portion of lever 210 extending into valve housing 222 is fitted with a membrane or stopper 230, which again may be made of a medically safe compressible (sealable) rubber, such as silicone. In the normally closed position of FIG. 3, where coil 204 is not energized and compression spring 208 is extended, lever 210 pivots membrane or stopper 230 so as to contact and seal closed an inner, e.g., beveled, port 226p of normally closed fluid outlet 226, thereby preventing fluid flow through the normally closed outlet. When coil 204 is energized, plunger 206 moves to the left so as to compress spring 208 and pivot lever 210, such that membrane or stopper 230 moves away from beveled port 226p of normally closed fluid outlet 226 to instead contact and seal closed an inner, e.g., beveled, port 228p of normally open fluid outlet 228, thereby preventing fluid flow through the normally open outlet. In the normally closed state, three-way valve 194 allows fluid, such as water or PD fluid, to flow from fluid inlet 224 through normally open fluid outlet 228. In the normally open state, three-way valve 194 allows fluid, such as water or PD fluid, to flow from fluid inlet 224 through normally closed fluid outlet 228.

Higher fluid pressure through fluid inlet 224 helps to seal membrane or stopper 230 against both normally closed port 226p and normally open port 228p, where the pressure in normally closed fluid outlet 226 and normally open fluid outlet 228 is less. It should be appreciated, however, that the pressure delta that helps to seal membrane or stopper 230 against beveled ports 226p, 228p, also fights against (i) the magnetic force induced when coil 164 is energized to open normally closed fluid outlet 226 and (ii) the force of compression spring 208 when coil 164 is de-energized to open normally open fluid outlet 226. Described herein is structure and associated functionality for ensuring that normally closed fluid outlet 226 is properly opened when it is commanded to be open.

### Solenoid Valve Methodology to Reduce Noise

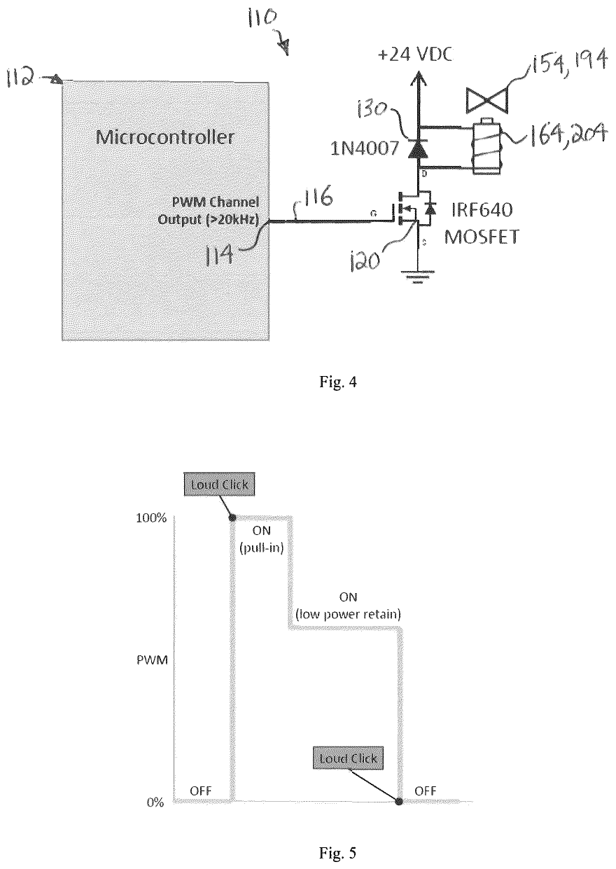

As previously discussed, there is a desire and need for reliable methodologies for reducing noise caused by solenoid valves in medical fluid delivery operations. Noise reduction is particularly pertinent for peritoneal dialysis systems, which operate close to a patient, and which may occur during the night when patients are sleeping and reduced noise is of the essence. A desirable PD system may be one in which the noise level is maintained below 33 decibels. FIG. 4 illustrates an example solenoid valve driving circuit 110 for the systems and associated methodology of the present disclosure, which may be used to lessen operational noise associated with solenoid valves.

Driving circuit 110 includes a programmable microcontroller 112, which is provided as part of control unit 100 (FIG. 1). Microcontroller 112 includes a pulse width modulation (“PWM”) channel output 114, which is in one embodiment greater than 20 KHz. A PWM drive waveform is delivered along line 116 to a metal oxide semiconductor field effect transistor (“MOSFET”) 120. While MOSFET 120 in the illustrated example may be an IRF640 MOSFET, MOSFET 120 may alternatively be any N-channel logic level gated MOSFET capable of handling greater than 323 mA. MOSFET 120 operates in combination with a diode 130 for powering coil 164 of two-way valve 154 or coil 204 of three-way solenoid valve 194. While diode 130 in the illustrated example may be a IN400X diode, similar diodes may be used instead. Notably, MOSFET 120 and diode 130 may be standard and relatively inexpensive components.

FIG. 5 illustrates an operation of solenoid valves 154, 194 without using driving circuit 110, where the PWM signal transitions instantaneously from zero to one-hundred percent to energize coil 164, 204 and instantaneously from one-hundred to zero percent to de-energize coil 164, 204. In both instances loud clicks or noises occur. Here, the PWM frequency is greater than 20 kXz, e.g., 78.1 kHz in one test.

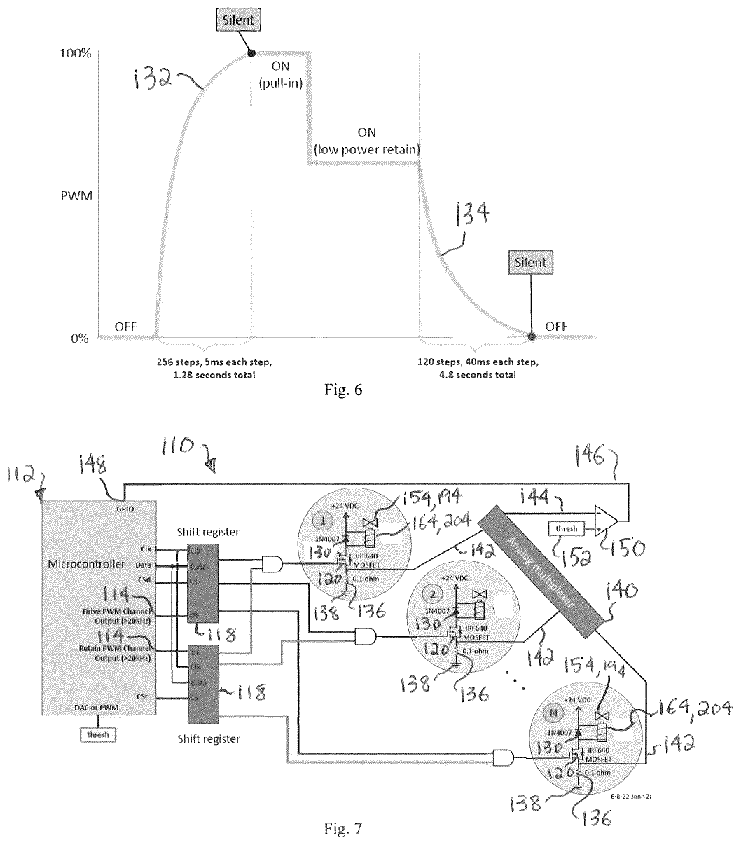

FIG. 6 illustrates operation of solenoid valves 154, 194 using driving circuit 110, which is programmed via software to follow a first curved PWM profile 132 from zero to one-hundred percent and a second curved PWM profile 134 from one-hundred zero percent. In both instances, the loud clicks or noises are eliminated. In the illustrated embodiment, a first curved PWM profile 132 is performed over 256 steps at 5 milliseconds per step, totaling 1.28 seconds to fully energize coil 164, 204. Additionally, in the illustrated embodiment, second curved PWM profile 134 is performed over 120 steps at 40 milliseconds per step, totaling 4.8 seconds to fully de-energize coil 164, 204. Here again, the PWM frequency is greater than 20 kHz, e.g., 78.1 kHz in one test. It should be appreciated from FIG. 6 that driving circuit 110 does not require positional feedback and that actuation and de-actuation are both quiet. Moreover, solenoid valves 154, 194 are opened and closed as positively as in FIG. 5, not using driving circuit 110.

FIG. 7 illustrates driving circuit 110 expanded to drive multiple coils 164, 204 of multiple solenoid valves 154, 194. Driving circuit 110 of FIG. 7 again includes programmable microcontroller 112, which is provided as part of control unit 100 (FIG. 1). Driving circuit 110 of FIG. 7 includes multiple, e.g., two, PWM channel outputs 114. PWM drive waveforms are delivered along lines 116 from PWM channel outputs 114 to shift registers 118. Shift registers 118 include cascading flip-flops that share a single clock signal, which causes the PWM drive waveforms to shift from one MOSFET 120, diode 130, and solenoid valve 154, 194 to the next, e.g., nth, MOSFET 120, diode 130, and solenoid valve 154, 194. FIG. 7 illustrates that driving circuit 110 may include a limited number of PWM channel outputs 114 to drive multiple solenoid valve 154, 194 so as to have greatly reduced noise output.

### Determining Position of the Solenoid Valve

As previously discussed, there is a desire and need for reliable methodologies for verifying the position of levers 170, 210 within solenoid valves 154, 194 to make sure a valve is not stuck or otherwise compromised. Knowing the position of levers 170, 210 also allows for self-calibration for the noise reduction PWM drive waveforms described above, where (i) a fully closed or zero percent PWM location of levers 170, 210 may be verified by microcontroller 110 prior to sending the noise reduction PWM drive waveform 132 to the solenoid valve, and (ii) a fully open or one-hundred percent PWM location of levers 170, 210 may be verified by microcontroller 110 prior to sending the noise reduction PWM drive waveform 134 to the solenoid valve. To obtain the position of levers 170, 210 within solenoid valves 154, 194, respectively, a resistor 136, e.g., 0.1 ohm resistor, is placed between MOSFET 120 and ground 138. Additionally, for each valve 154, 194 a position detection line 142 is extended from a point between MOSFET 120 and resistor 136 to an analog multiplexer 140. Multiplexer 140 selects between multiple analog signals traveling along position detection lines 142 for outputting to a single output line 144 extending from multiplexer 140. Multiplexer 140 makes it possible for multiple valves 154, 194 to share a single comparator 150.

Comparator 150 is configured to compare an analog position detection signal traveling along position detection line 142 from one of solenoid valves 154, 194 to a threshold signal 152. The comparison by comparator 150 leads to an output indicative of the position of lever 170, 210 within solenoid valve 154, 194, which is delivered along a comparator output line 146 to a general purpose input/output (GPIO) port 148 of programmable microcontroller 110, which uses programmed software to analyze the comparator output to thereby know/verify the position of lever 170, 210 within solenoid valve 154, 194 via the sampling of each valve via multiplexer 140.

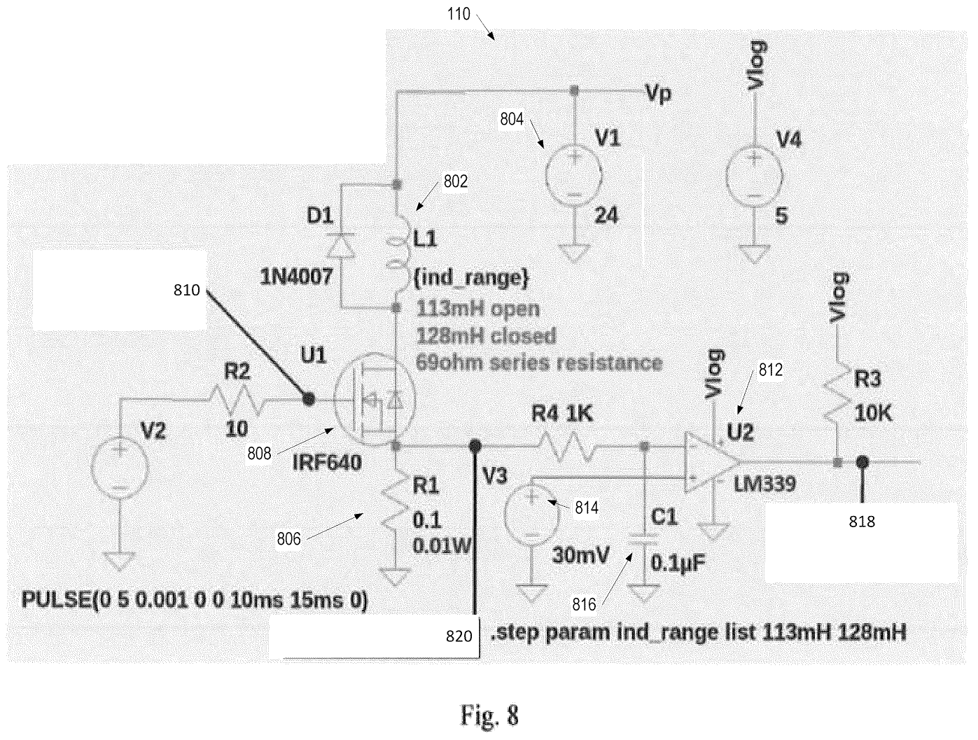

FIG. 8 is an electrical schematic illustrating the driving circuit 110 for determining the position of a valve lever in a given solenoid valve, according to an example embodiment of the present disclosure. As shown in FIG. 8, the driving circuit 110 includes solenoid inductor coils 802 driving a valve. The current to drive the solenoid inductor coils 802 may be provided via a voltage supply 804 (e.g., 24 V). A sense resistor 806 may be used to measure current 820 (e.g., by dividing the voltage by the resistance of the sense resistor 806) that drives the solenoid inductor coils 802. For example, the current 820 passing through the sense resistor 806 may be substantially the same as the current passing through the solenoid coil inductor 802 (e.g., to trigger the solenoid valve). Thus, if a voltage were to be measured across the sense resistor 806, that voltage would be proportional to the current 820. In the example driving circuit 110 shown in FIG. 8, the sense resistor 806 exhibits a resistance of 0.1 Ohms.

The current 820 to drive the solenoid coils may pass through transistor 808 (e.g., MOSFET (IRF640))) on the command of a microcontroller (e.g., microcontroller 112). For example, a pulse width modulation (PWM) waveform may be generated by associated PWM hardware in the microcontroller. The PWM waveform may drive a gate 810 of the transistor 808. The current 820 that enters the solenoid coil valve 802 may be measured using the sense resistor 806. In some embodiments, the current 820 may be filtered using an RC filter 816 (e.g., a low pass filter) comprising, for example, a capacitor and a resistor. The current 820 may enter the comparator 812. The comparator 812 is configured to compare the voltage associated with the above described current 820 with a threshold voltage supplied by a threshold voltage supply 814. For example, as shown in FIG. 8, the threshold voltage may be 0.7 V. When the voltage associated with the current 820 passing through the solenoid coil transistor 802 is higher than the threshold voltage 814, the comparator 812 is configured to produce a digital output (e.g., a “high” or “1” signal) that is sent to the microcontroller (e.g., microcontroller 112) via a GPIO line 818. The voltage associated with the current 820 may be used to generate a waveform that is discussed herein, e.g., in relation to subsequent figures. In some embodiments, the threshold voltage may be configured to be suitable to voltage ranges expected of the current 820, which may depend on the resistance value of the sense resistor 806 that is placed in the driving circuit 110.

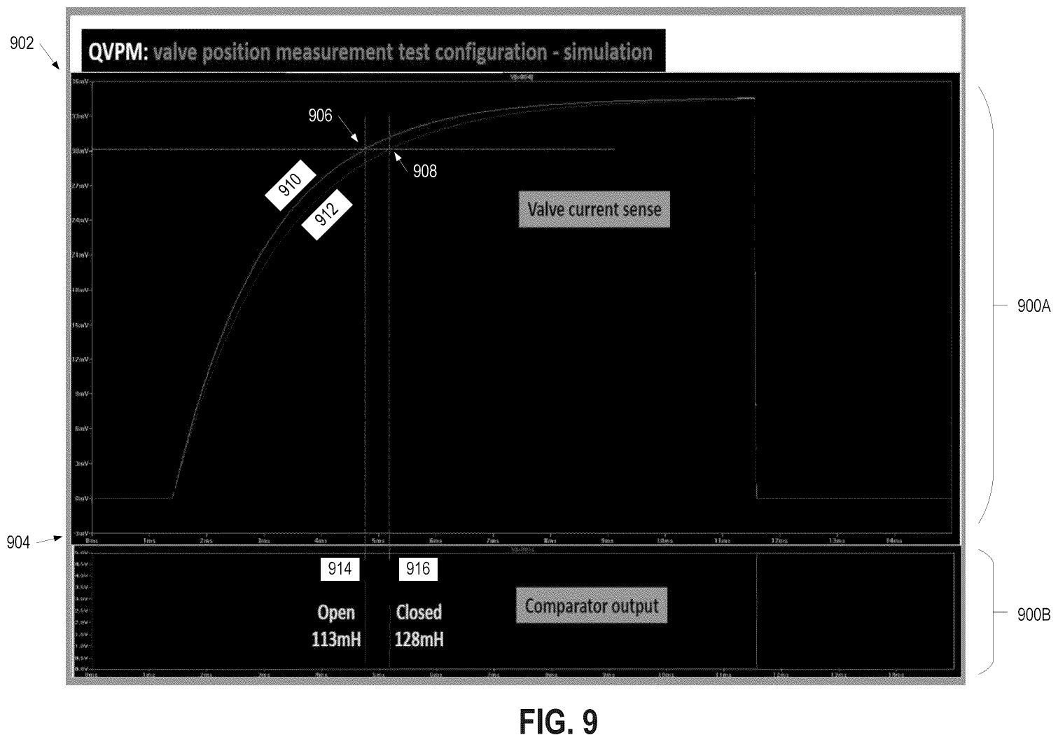

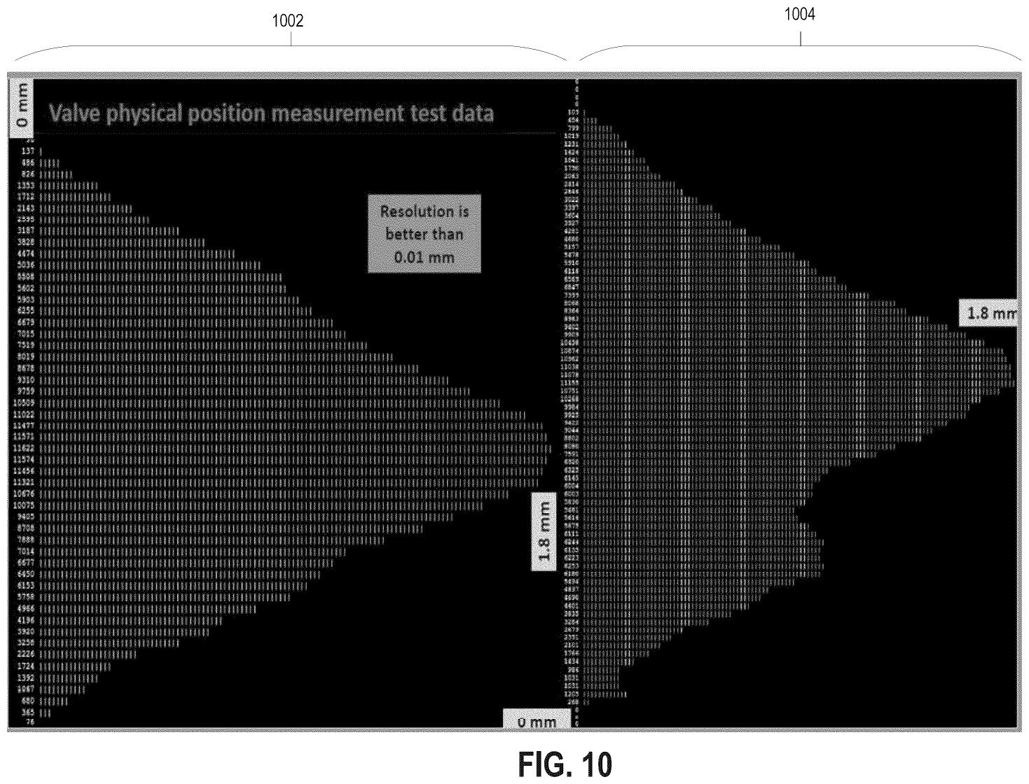

FIG. 9 is a graphical output from a valve lever 170, 210 position test configuration, which illustrates a position of a valve based on inductance, according to an example embodiment of the present disclosure. Specifically, FIG. 9 shows two graphs: a top graph 900a illustrates a voltage 902 on a y-axis, which is based on current 820 that is sensed in the driving circuit 110 (e.g., based on the resistance of sensor resistor 806) over time 904, which is shown on a x-axis; and a bottom graph 900b, which illustrates a digital output of the comparator 812 over the same time range. It is contemplated that a position of a plunger (e.g., a metal slug) within a solenoid coil, which is indicative of the position of a valve, reflects an inductance of the solenoid coil. Thus, by measuring an inductance of the solenoid coil, one may determine the extent to which the plunger is within the solenoid coil (e.g., whether the plunger is all the way in, whether it is all the way out, or somewhere in the middle). For example, the plunger being fully inside the solenoid coil may yield a higher inductance than when the plunger is outside or not fully inside. When the valve is open (e.g., and not actuated), less of the plunger may be inside the magnetic field induced by the solenoid coil, (since the plunger may be partially outside of the solenoid coil), causing the inductance to be lower. However, since inductance is lower, the magnetic field may build up faster, causing the current flowing through solenoid coil to change more quickly with time. Since the current correlates with the voltage (e.g., based on the sense resistor 806), the voltage changes more quickly with time, causing the voltage to reach a given threshold voltage (e.g., 30 mV) relatively sooner in time. In contrast, when the solenoid valve is closed, then more of the plunger may be located inside the solenoid coil and therefore more inside the magnetic field induced by the solenoid coil. This presence may increase the inductance, causing the current to change more slowly. Since the current correlates with the voltage (e.g., based on the sense resistor 806), the voltage changes more slowly with time, causing the voltage to reach the given threshold voltage (e.g., 30 mV) relatively later in time.

As shown in graph 900a, curve 910 represents the voltage over time for the scenario where the valve is open (e.g., and not actuated), whereas curve 912 represents the voltage over time for the scenario where the valve is closed. As discussed above, curve 910 shows the voltage associated with when the valve is open, reaches a given threshold voltage of 30 mV much sooner in time 906 than the time 908 at which curve 912 reaches the same threshold voltage. The difference is thus a result of the difference in the position of the plunger within the solenoid coil, which results in different inductance levels, which results in differences in time taken by the solenoid coil to reach the threshold voltage. Thus, the difference between times 906 and 908 may reflect a change in inductance. For the particular valve associated with the graphical results shown in FIG. 9, when the valve is open (e.g., when the plunger is not fully inside the magnetic field of the solenoid coil), the inductance of the valve is 113 mH. However, when the valve is closed (e.g., the plunger is drawn further inside the solenoid coil), the inductance of the valve is 128 mH. The difference in times 906 and 908 may thus correspond to the difference between these inductances (113 mH and 128 mH). By measuring the time precisely, one may be able to differentiate between an open valve and closed valve.

As shown in graph 900b, the digital output of the comparator of the driving circuit 110 associated with the above described valve shows curves 914 and 916 that correspond to the curves 910 and 912, respectively. Curve 914 of graph 900b, which represents the digital output of the comparator of an opened valve, shows that the comparator yields a high signal (e.g., a true or “1” signal) when the voltage associated with the solenoid coil that is opened crosses the voltage threshold. Curve 916 of graph 900b, which represents the digital output of the comparator of a closed valve, yields a low signal (e.g., a false or “0” signal) when the voltage associated with the solenoid coil that is opened crosses the voltage threshold. However, as there is a difference in inductance associated with an open valve as opposed to a closed valve (e.g., 113 mH versus 128 mH, respectively), which results in a difference in time for an applied voltage to reach a voltage threshold, there is a difference in time at which the digital output of the comparator shifts its signal for the respective curves (e.g., as shown in graph 900B).

FIG. 10 is a data output from a valve lever 170, 210 position test configuration, which illustrates the position of a plunger in the valve over time, in accordance with a non-limiting embodiment of the present disclosure. For example, graph 1002 of FIG. 10 shows data output indicating the position of the plunger after a valve is conventionally opened or conventionally closed, and graph 1004 of FIG. 10 shows data output indicating the position of the plunger after a valve has been opened or closed using the previously discussed systems and methodologies to reduce noise. In these graphs 1002 and 1004, the vertical axis represents the time while the horizontal axis represents the position of the plunger (e.g., from 0 mm to 1.8 mm). The graphs show, to a high resolution, that the plunger may move a distance of 1.8 mm to actuate the valve. Each line is a sample point at a given time, with the displacement represented by the number of digits. As shown by the graphs in FIG. 10, the systems and methods presented herein may allow one to determine the position of the valve (e.g., via the position of the plunger in the valve) with a high degree of resolution (e.g., better than a hundredth of a millimeter).

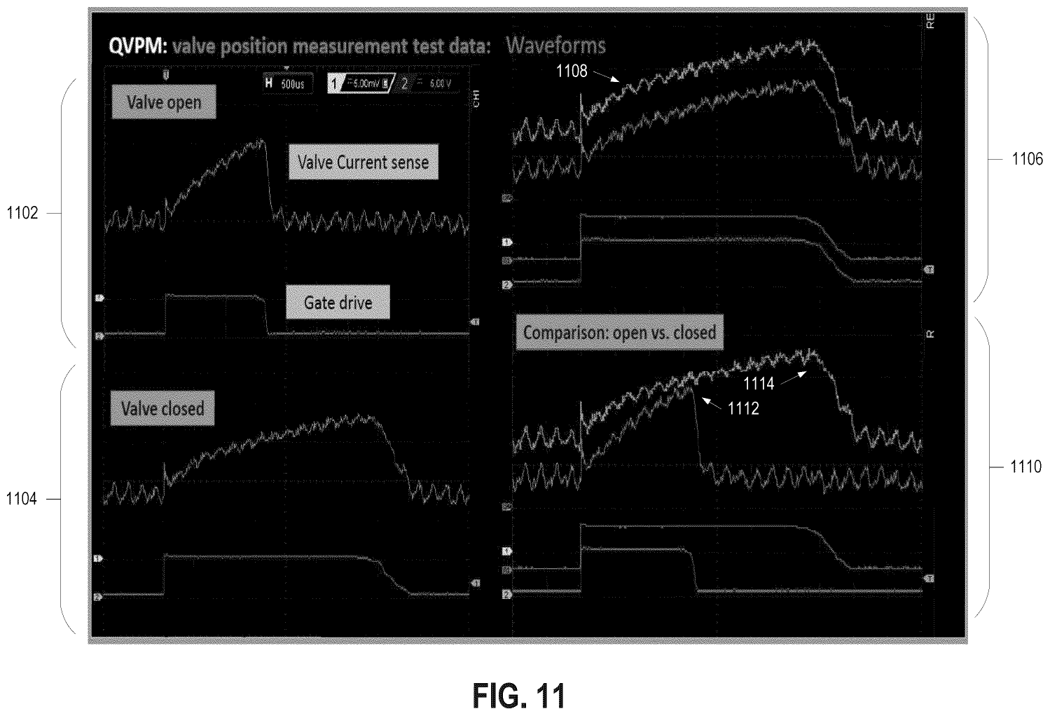

FIG. 11 illustrates various waveforms from a valve lever 170, 210 position test, which illustrate measured voltage over time to determine the position of the valve when a valve is open and when a valve is closed, in accordance with non-limiting embodiments of the present disclosure. For example, graphs 1102 and 1104 present voltage measured at intersection (e.g., current 820 of FIG. 8) of the driving circuit 110, coupled with the voltage 810 provided to the gate of the transistor 808 (e.g., gate drive). While graph 1102 shows the measured voltage over time to determine the actual position of the valve, when the valve is reportedly in an open state, graph 1104 shows the measured voltage over time to determine the actual position of the valve, when the valve is reportedly in a closed state. Furthermore, graph 1106 overlays graph 1104 depicting the actual measured voltage over time for a valve that is reportedly in a closed state with a reference waveform 1108 of a reference voltage over time for a valve that is actually in a closed state. Graph 1110 overlays the reference waveform of a reference voltage measured over time for a valve that is actually in an open state, with the reference waveform 1108 of the reference voltage over time for a valve that is actually in a closed state. For each of these graphs, the measured voltages may be based on the sense resistor 806 used in the driving circuit 110. In some aspects, the waveforms may be measured or output by a processor or microprocessor (e.g., at microcontroller 112).

The difference in time between edge 1112 and edge 1114 (e.g., the difference in time taken to reach the threshold voltage) is indicative of a valve being open or the valve being closed, respectively. As previously discussed, when a valve is open (e.g., when a plunger is partially outside of a magnetic field of a solenoid coil associated with the valve), the inductance of the solenoid coil may be at its lowest point, causing the current level of the current entering the valve to rise more quickly until it reaches a threshold voltage. Since the current is proportionate to the measured voltage (e.g., based on the sense resistor 806), the waveform for a valve that is open thus shows a quicker rise of the measured voltage to the threshold voltage. This quicker rise is shown, for example, in graph 1102, and in edge 1112. When the valve is closed, the plunger may be located further into the magnetic field of the solenoid coil, causing the current to rise more slowly. Thus, as shown in graphs 1104 and 1106, the measured voltage (e.g., across sense resistor 806) may take longer to reach the same threshold voltage.

For a valve whose actual state (e.g., open or closed) or position of the plunger is yet to be determined or confirmed, a voltage may be similarly measured (e.g., using a sense resistor 806 as used in the driving circuit 110). The microcontroller 112 may search for a transition in the measured voltage (e.g., as in the edges 1112 and 1114) in the waveform of the measured voltage. The transition may be the point at which the measured voltage reaches the threshold voltage. The microcontroller 112 may determine the time the valve takes to reach that transition (e.g., the threshold voltage). The position of the plunger within the valve, and thereby the state of the valve, may be determined by identifying whether the time to reach the transition is closer to the time it takes to reach edge 1114 (e.g., in which case the valve may be closer to being in a closed state) or closer to the time it takes to reach edge 1112 (e.g., in which case the valve may be closer to being in the open state). The precise point of the plunger may thus be determined based on the time it takes the valve to reach the threshold voltage and comparing the time to known times for reaching the threshold voltage in the closed and open states. For example, if the time it takes the measured voltage in the valve to reach the threshold voltage is halfway between the times to reach edges 1112 and 1114, then the position of the plunger in the valve is halfway between fully inside and fully outside, leaving the valve neither open nor closed (i.e., the valve is stuck). In some embodiments, the microcontroller 112 may perform image processing on the measured voltage of the valve to obtain a clearer waveform in order to better identify the point of transition (e.g., the time it takes for the measured voltage to reach the threshold voltage).

The microcontroller 112 is configured to provide information indicative of whether the valve is in the commanded position. In some embodiments, the microcontroller 112 may generate an alarm (e.g., via the user interface 108) after detecting that a valve is not in a commanded position. Additionally or alternatively, the microcontroller 112 may pause a dialysis treatment. In some instances, the microcontroller 112 may attempt to re-actuate the valve a number of times to get the valve to move to the commended position. When the valve position is still not correct, the microcontroller 112 may then generate an alarm.

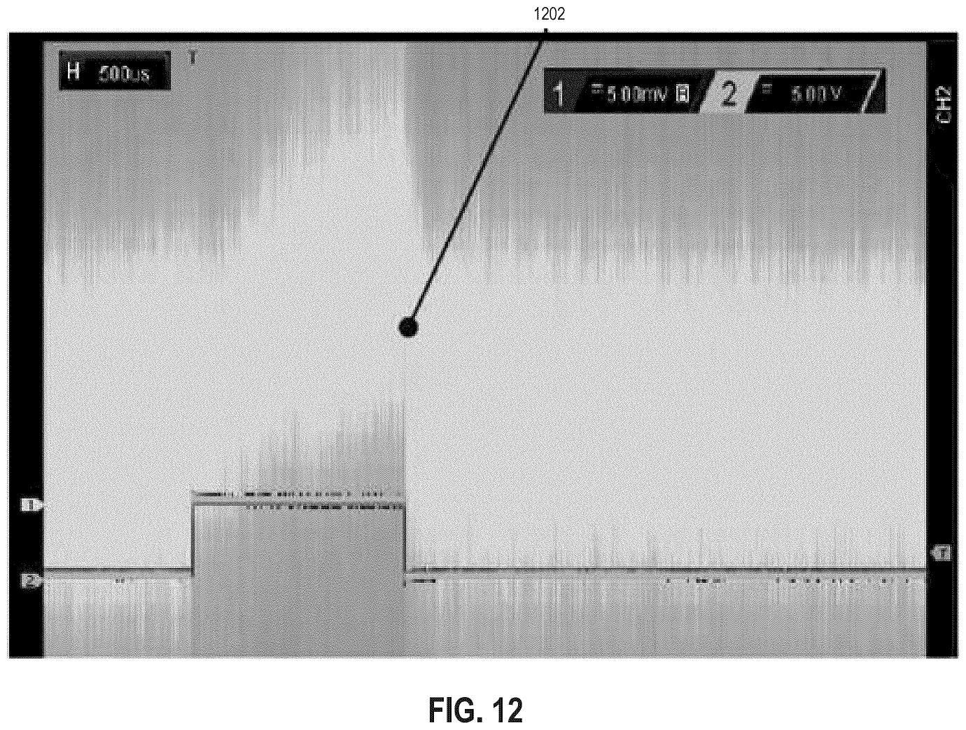

FIG. 12 illustrates an averaged waveform from multiple valve lever 170, 210 position tests performed on a valve, which show a characteristic transition point 1202 for that valve. The transition point 1202 may indicate the averaged time at which the averaged measured voltage from the valve reaches a threshold voltage. As shown in FIG. 12, the averaging of multiple waveforms results in a very noisy averaged waveform. For example, the individual pulses of the various duty cycles in the averaged waveform may not be as discernable. Even then, the transition point 1202 may be easily identifiable and can be used to determine the valve position to a high resolution (e.g., at least better than 0.01 mm resolution). Furthermore, the various duty cycles may determine how much power in aggregate goes to the valve to generate the waveform of the measured voltage

As will be discussed herein, in relation to subsequent figures, measuring the position of the valve when the valve is not actuated (e.g., when the valve is not being powered) may involve different preprocessing steps than when the valve is actuated, because the solenoid coil associated with the valve can be saturated and that saturation can have an effect on the waveform of the measured voltage.

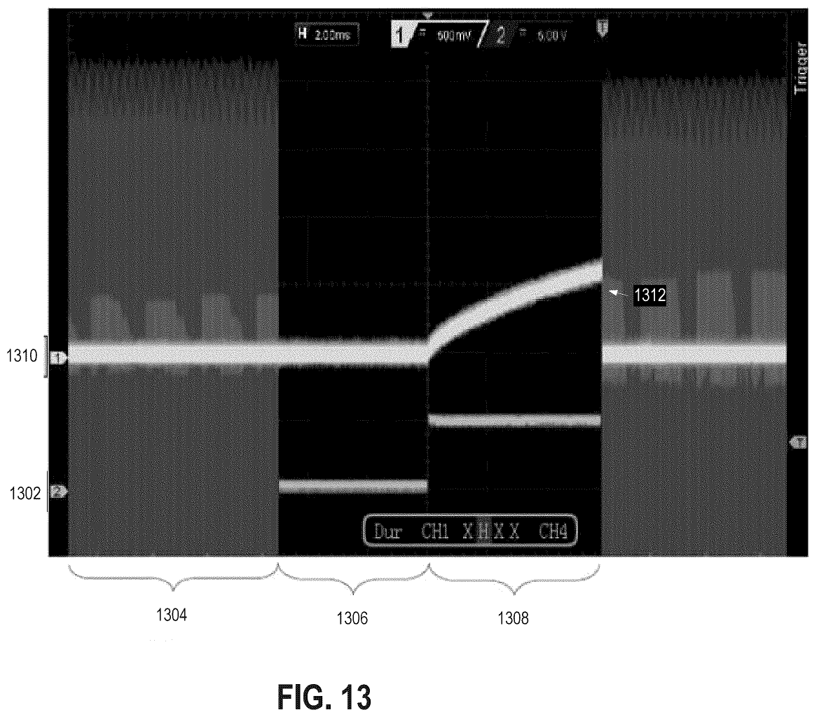

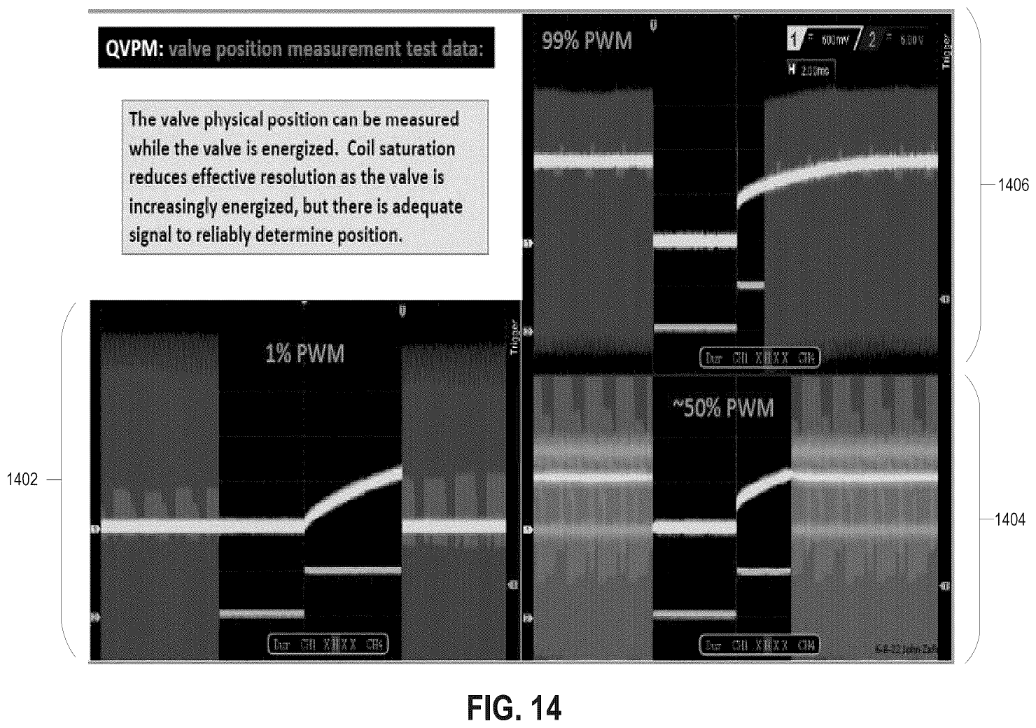

FIGS. 13 and 14 illustrate valve lever 170, 210 position test data recorded after the gate drive has been desaturated, in accordance with a non-limiting embodiment of the present disclosure. In some embodiments, in order to effectively assess a waveform output of a measured voltage of a valve, the microcontroller 112 may need to temporarily turn off the pulse width modulation (PWM), causing the power supplied to the driving circuit 110 to fall to zero for a predetermined period of time (e.g., 5 ms). The plunger associated with the valve may be prevented from or rendered incapable of moving anywhere in that predetermined period (e.g., due to the mass of the plunger). Moreover, the microcontroller 112 may turn off the power during the predetermined period in order to desaturate the solenoid coil associated with the valve. The predetermined period may be long enough for the solenoid coil to be able to make useful measurements during the measurement interval by minimizing noise caused by saturation, but not long enough to release the valve or cause the valve to chatter. After the predetermined period passes, the microcontroller 112 may reapply power to the valve (e.g., by causing current to flow to the gate of the transistor 808).

Thus, as shown in FIG. 13, when a given valve's state and position of the plunger associated with the valve is to be measured, the microcontroller 112 may cause the power applied by gate drive 1302 to go to zero from its previous phase 1304, where power may have been applied to drive the valve via pulse width modulation. The precise power previously being applied to the valve may vary, resulting in different waveforms in phase 1304, as will be discussed in relation to FIG. 14. Subsequently, the gate drive may reduce and/or apply zero power to the valve during a desaturation phase 1306 lasting a predetermined period of time (e.g., 5 ms). After the predetermined period of time passes, the valve may be ready (e.g., desaturated) for its voltage to be measured (e.g., over measurement interval 1308). The corresponding waveform 1310 of the measured voltage, including the transition point 1312 is also shown. The measurement interval 1308 may thus be the time it takes for the measured voltage of the valve to reach the transition point 1312 (i.e., the threshold voltage).

FIG. 14 shows valve lever 170, 210 position test data recorded after the gate drive has been desaturated, where the valve was previously applied with power at different levels. For example, graph 1402 illustrates the resulting waveform when the microcontroller 112 applied PWM at 1%, graph 1404 illustrates the resulting waveform when the microcontroller 112 applied PWM at 50%, and graph 1406 illustrates the resulting waveform when the microcontroller 112 applied PWM at 99%. The yellow trace throughout graphs 1402-1406 and in FIG. 13 is the measured voltage across the valve (e.g., based on the measured voltage across sense resistor 806) and may thus be indicative of the current flowing through the valve. For each previously existing PWM level (e.g., graphs 1402-1406), the current being applied went to 0 Amps during the respective desaturation phases (e.g., desaturation phase 1306), and then rose during the respective measurement intervals (e.g., measurement interval 1308).

When the rising current causes the voltage applied across the valve to reach the threshold voltage, the comparator 812 may provide a signal to indicate this transition point (i.e., the point at which the measured voltage reaches the threshold voltage). The microcontroller 112 may thus measure the time it takes for the voltage to reach the voltage threshold by determining at what time the comparator 812 sends the signal after the measurement interval 1308 begins. For example, the comparator 812 may send a low to high digital signal to the microcontroller 112 via the IO line 818 once the valve reaches the transition point, and the microcontroller 112 may detect the low to high signal and may end a timer to mark the time incurred during the measurement interval, in response to the detection. The microcontroller 112 may thereafter revert back to driving whichever PWM duty cycle it was set to drive the valve with before (e.g., in the previous phase 1304).

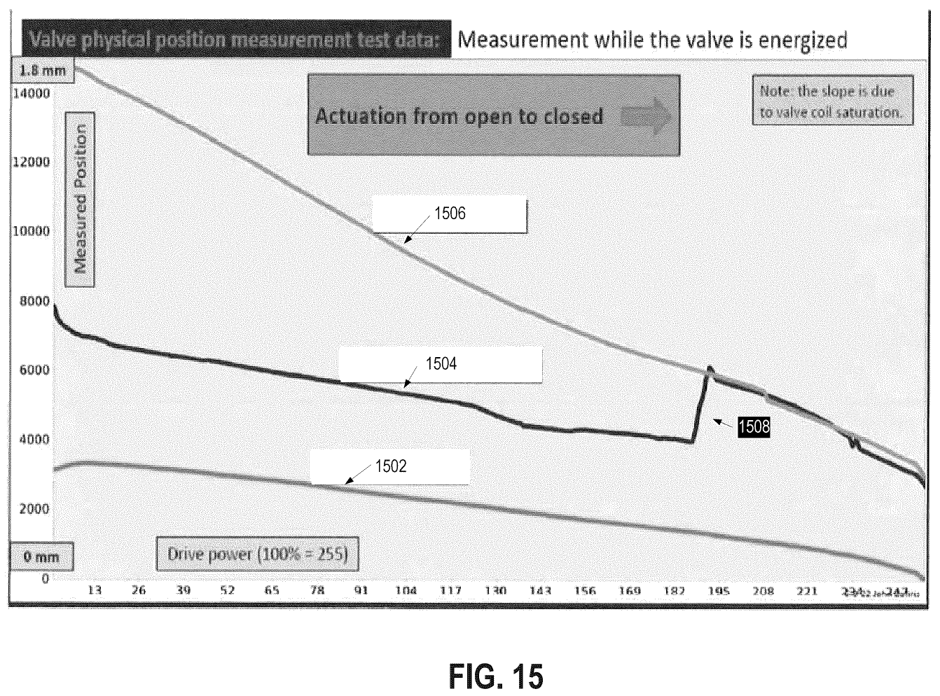

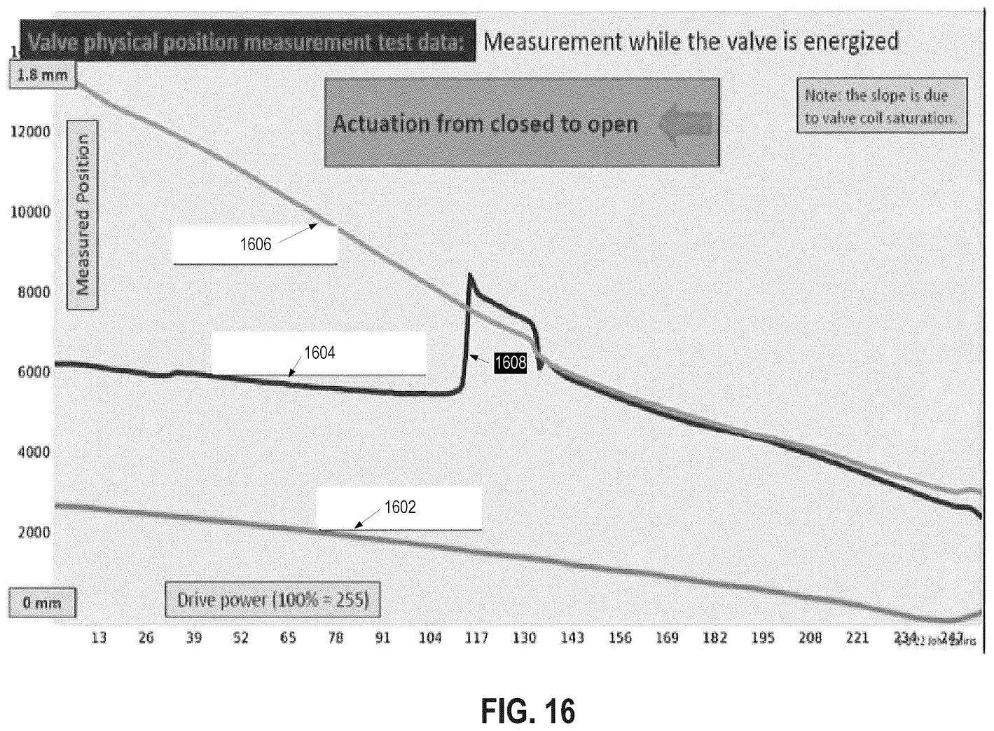

FIGS. 15 and 16 illustrate valve lever 170, 210 position test data recorded showing valve lever 170, 120 being stuck closed, valve lever 170, 120 acting normally, and valve lever stuck open. Specifically, FIG. 15 illustrates the three above-described position test data for a valve that is commanded to go from an open to a closed state even though only one of the three position test data (i.e., position test data 1504) illustrates the valve actually actuating normally in response to the command. Similarly, FIG. 16 illustrates the three above-described position test data for a valve that is commanded to go from a closed to an open state even though only one of the three position test data (i.e., position test data 1604) illustrates the valve actually actuating normally in response to the command. Such position test data are based on inductance measurements of the valve as it is commanded to actuate.

As shown in FIGS. 15 and 16, each position test data 1502-1506, 1602-1606 is sloped downward as a result of saturation in the solenoid coil associated with the valve. It is to be appreciated that if the valve was not being actuated at all, such position test data 1502-1506, 1602-1606 may be largely horizontal (e.g., have little to no slope). However, since the position test data is being received when the valve is commanded to actuate (e.g., from open to a closed state in FIG. 15 and from a closed to an open state in FIG. 16), the solenoid coil associated with the valve begins to saturate, causing the downward slopes in position test data 1502-1506, 1602-1606. As previously discussed, the microcontroller 112 can mitigate the noise caused by the saturation during the measurement interval 1308 by undergoing a desaturation phase 1306 where the power being applied to the valve is set to zero for a predetermined period of time. However, even with the downward slope caused by the saturation, FIGS. 15 and 16 quite clearly illustrates when a valve is operating normally (e.g., position test data 1504 and 1604), as opposed to being stuck open (e.g., position test data 1502 and 1602) or stuck closed (e.g., position test data 1506 and 1606). Furthermore, FIG. 15 shows the actual transition 1508 of a valve from an open to a closed state, and FIG. 16 shows the actual transition 1508 of a valve from a closed state to an open state, when the valves in both scenarios operate normally. However, while the position test data are to be traced from left to right in FIG. 15, as the valve is commanded to close, the position test data are to be traced from right to left in FIG. 16, as the valve is commanded to open. Thus, the respective transition points 1508 and 1608, may occur at different positions and/or times depending on whether the valve is commanded to open (e.g., FIG. 15) or close (e.g., FIG. 16). Furthermore, the respective transition points 1508 and 1608 could vary based on the particular characteristics of the valve (e.g., the manufactured batch), or may change over time as the valve wears.

Therefore, in some embodiments, a generic waveform may be used to compare the measured voltage of a valve (e.g., based on sense resistor 806) to a generic threshold voltage. The generic waveform may be customized so that the transition point of the measured voltage from the valve aligns with the threshold voltage asymptotically.

It should be understood that various changes and modifications to the presently preferred embodiments described herein will be apparent to those skilled in the art. It is therefore intended that such changes and modifications be covered by the appended claims. For example, while the MOSFET and NPN transistors are described as transistors used to assist in noise reduction during solenoid valve activation and deactivation, other transistors may similarly be used to slow down the plunger in the solenoid valves to similarly reduce noise. In another example, while different valve embodiments have been discussed primarily in connection with peritoneal dialysis (“PD”), the valve embodiments may be used with other medical fluid systems and associated machines, such as ones for hemodialysis (“HD”), hemofiltration (“HF”), hemodiafiltration (“HDF”), and continuous renal replacement treatment (“CRRT”).

The invention is claimed as follows:

1. A medical fluid system comprising:
a valve configured to control a fluid flow in the medical fluid system, wherein the valve comprises a housing, a solenoid coil, and a plunger, and wherein the valve is configured to activate a flow of fluid through a tube by applying a voltage to the solenoid coil to move the plunger within the housing;
a driving circuit configured to control the valve of the medical fluid system via pulse width modulation (PWM) signals, in response to control signals; and
a microcontroller comprising a processor and a memory, wherein the memory stores instructions that, when executed by the processor, cause the processor to:
transmit, to the driving circuit, a control signal causing the driving circuit to apply and ramp up a PWM signal to the solenoid coil of the valve,
wherein the ramping up of the PWM signal causes the plunger to move slowly until it reaches an end position within the housing, and
wherein the plunger moving slowly reduces sound generated by the plunger.

2. The medical fluid system of claim 1, wherein the instructions, when executed by the processor, further cause the processor to:
apply, via the driving circuit, power to the valve to measure a voltage across the valve;
monitor, via the driving circuit based on a sense resistor associated with the valve, the voltage across the valve over a measurement interval, wherein the measurement interval terminates when the voltage reaches a predetermined threshold voltage;
compare the measurement interval to a reference interval for a normally functioning closed valve; and
generate, based on the comparison of the measurement interval to the reference interval, an assessment of the valve position.

3. The medical fluid system of claim 1, wherein the valve is further configured to close the flow of medical fluid through the tube by un-applying, via the driving circuit, the voltage to the solenoid coil to move the plunger in an opposite direction from the housing to occlude the tube, wherein the instructions, when executed by the processor, further cause the processor to:
transmit, to the driving circuit, a second control signal causing the driving circuit to ramp down the PWM signal to the solenoid coil to a duty cycle of 0%,
wherein the ramping down of the PWM signal causes the plunger to move in an opposite direction and in a manner in which sound generated by the corresponding plunger is reduced in comparison to operation without the PWM signal.

4. The medical fluid system of claim 3, wherein the instructions, when executed by the processor, further cause the processor to:
apply, via the driving circuit, second power to the valve to measure a second voltage across the valve;
monitor, via the driving circuit based on a sense resistor associated with the valve, the second voltage across the valve over a second measurement interval, wherein the second measurement interval terminates when the second voltage reaches the predetermined threshold voltage;
compare the second measurement interval to a second reference interval for a normally functioning open valve; and
generate, based on the comparison of the second measurement interval to the second reference interval, a second assessment of the valve position.

5. The medical fluid system of claim 1, wherein the valve, the driving circuit, and the microcontroller are included within a peritoneal dialysis machine or a hemodialysis machine.

6. A method of determining valve position in a medical fluid system, the method comprising:
transmitting, by a microcontroller having a processor, a control signal for closing a valve, wherein the valve is configured to control a fluid flow in the medical fluid system, wherein the valve is open at least before the control signal is transmitted, and wherein the valve comprises a housing, a solenoid coil, and a plunger;
applying, via a driving circuit, power to the valve to measure a voltage across the valve;
monitoring, via the driving circuit based on a sense resistor associated with the valve, the voltage across the valve over a measurement interval, wherein the measurement interval terminates when the voltage reaches a predetermined threshold voltage;
comparing the measurement interval to a reference interval for a normally functioning closed valve; and
generating, based on the comparison, information indicative of whether the valve is closed.

7. The method of claim 6, further comprising, prior to applying the power to the valve,
setting a power level to zero for a predetermined period of time to cause desaturation of the solenoid coil over the predetermined period of time.

8. The method of claim 7, wherein the predetermined period of time is 5 milliseconds (ms).

9. The method of claim 6, wherein monitoring the voltage across the valve comprises:
receiving, from a comparator connected to the driving circuit, an indication of when the voltage reaches the predetermined threshold voltage.

10. The method of claim 6, further comprising:
transmitting, by the microcontroller, a second control signal for opening the valve;
applying, via the driving circuit, second power to the valve to measure a second voltage across the valve;
monitoring, via the driving circuit based on the sense resistor, the second voltage across the valve over a second measurement interval, wherein the second measurement interval terminates when the second voltage reaches a second predetermined threshold voltage;
comparing the second measurement interval to a second reference interval for a normally functioning open valve; and
generating, based on the comparison between the second measurement interval with the second reference interval, second information indicative of whether the valve is open.

11. A medical fluid system comprising:
a valve configured to control a fluid flow in the medical fluid system, wherein the valve comprises a housing, a solenoid coil, and a plunger, and wherein the valve is configured to activate a flow of fluid by applying a voltage to the solenoid coil to move the plunger within the housing;
a driving circuit configured to control the valve of the medical fluid system via pulse width modulation (PWM) signals, in response to control signals; and
a microcontroller comprising a processor and a memory, wherein the memory stores instructions that, when executed by the processor, cause the processor to:
transmit a control signal for closing the valve, wherein the valve is open at least before the control signal is transmitted,
apply, via the driving circuit, power to the valve to measure a voltage across the valve,
monitor, via the driving circuit based on a sense resistor associated with the valve, the voltage across the valve over a measurement interval, wherein the measurement interval terminates when the voltage reaches a predetermined threshold voltage,
compare the measurement interval to a reference interval for a normally functioning closed valve, and
generate, based on the comparison, information indicative of whether the valve is closed.

12. The medical fluid system of claim 11, wherein the instructions, when executed by the processor, further cause the processor to:
before applying the power to the valve, set a power level to zero for a predetermined period of time to cause desaturation of the solenoid coil over the predetermined period of time.

13. The medical fluid system of claim 12, wherein the predetermined period of time is 5 milliseconds (ms).

14. The medical fluid system of claim 11, wherein monitoring the voltage across the valve comprises receiving, from a comparator connected to the driving circuit, an indication of when the voltage reaches the predetermined threshold voltage.

15. The medical fluid system of claim 11, wherein the instructions, when executed by the processor, further cause the processor to:
transmit a second control signal for opening the valve;
apply, via the driving circuit, second power to the valve to measure a second voltage across the valve;
monitor, via the driving circuit based on the sense resistor, the second voltage across the valve over a second measurement interval, wherein the second measurement interval terminates when the second voltage reaches a second predetermined threshold voltage;
compare the second measurement interval to a second reference interval for a normally functioning open valve; and
generate, based on the comparison between the second measurement interval with the second reference interval, second information indicative of whether the valve is open.

16. The medical fluid system of claim 11, wherein the valve, the driving circuit, and the microcontroller are included within a peritoneal dialysis machine or a hemodialysis machine.

17. A method of controlling a valve, the method comprising:
transmitting, by a microcontroller having a processor, a control signal for causing a driving circuit to apply and ramp up a PWM signal to a solenoid coil of a valve, wherein the valve is configured to activate a flow of fluid by applying a voltage to the solenoid coil to move a plunger within a housing,
wherein the ramping up of the PWM signal causes the plunger to move slowly until it reaches an end position within the housing, and
wherein the plunger moving slowly reduces sound generated by the plunger.

18. The method of claim 17, further comprising:
applying, via the driving circuit, power to the valve to measure a voltage across the valve;
monitoring, via the driving circuit based on a sense resistor associated with the valve, the voltage across the valve over a measurement interval, wherein the measurement interval terminates when the voltage reaches a predetermined threshold voltage;
comparing the measurement interval to a reference interval for a normally functioning closed valve; and
generating, based on the comparison of the measurement interval to the reference interval, an assessment of the valve position.

19. The method of claim 17, wherein the valve is further configured to close the flow of medical fluid by un-applying, via the driving circuit, the voltage to the solenoid coil to move the plunger in an opposite direction from the housing to occlude the tube, the method further comprising:
transmitting, by the microcontroller to the driving circuit, a second control signal causing the driving circuit to ramp down the PWM signal to the solenoid coil to a duty cycle of 0%,
wherein the ramping down of the PWM signal causes the plunger to move in an opposite direction and in a manner in which sound generated by the corresponding plunger is reduced in comparison to operation without the PWM signal.

20. The method of claim 19, further comprising:
applying, via the driving circuit, second power to the valve to measure a second voltage across the valve;
monitoring, via the driving circuit based on a sense resistor associated with the valve, the second voltage across the valve over a second measurement interval, wherein the second measurement interval terminates when the second voltage reaches the predetermined threshold voltage;
comparing the second measurement interval to a second reference interval for a normally functioning open valve; and
generating, based on the comparison of the second measurement interval to the second reference interval, a second assessment of the valve position.
